(19) United States
(12) Patent Application Publication
(10) Pub. No.: US 2004/0087840 A1
(43) Pub. Date: May 6, 2004

(54) Multiple function birth assistance appliance

(76) Inventor: David Roy Main, Boulder Creek, CA (US)

Correspondence Address: David R. Main, 181 Oak Knoll Dr., Boulder Creek, CA, 95006, US

(21) Appl. No.: 10/286,515

(22) Filed: Oct. 31, 2002

(51) Int. Cl. A61B 5/00

(52) U.S. Cl. 600/304

(57) ABSTRACT

An integrated function appliance to assist the process of child birth by providing convenient timing of labor contractions, to calculate contraction event parameters, to compare contraction parameter values to pre-defined values and alert the user to the probable immediacy of birth based upon the comparison, to provide pain management assistance with a visual focus object which may be synchronized to contraction events, to provide pain management assistance with an ergonomically fashioned handle suitable for vigorous labor pain response squeezing. The invention may use a single variable capacitance actuated control input means. The invention is ergonomically designed to be is non-intrusive in the stressful context of the labor process.

### RELATED PATENTS

Related Patents

| | |
|---|---|
| 4,047,010 | September 1977 Perotto |
| 4,493,043 | January 1985 Forbath |
| 4,497,312 | Februay 1985 Byrd |
| 5,042,503 | August 1991 Torok |
| 5,876,335 | March 1999 Handy |

### STATEMENT OF FEDERALLY SPONSORED RESEARCH

Not applicable

### REFERENCE TO MICROFICHE

Not applicable

### BACKGROUND OF THE INVENTION

Child birth is an extremely demanding experience for a mother and those who assist with the process. The gestation period climaxes with a sequence of events which vary from birth to birth but which typically exhibit some similarities. Excluding abnormal births, some birth similarities are the early, intermediate, and active labor phases with significant physical stress for the mother due to intense pain. There is also the mother's need to avoid unwelcome physical and emotional irritants, to concentrate without superfluous distractions, for confidence that the labor process is progressing and her pain will end, and for an emotional sense of having some control over her situation (i.e. not being helpless). Many mothers believe it is desirable to self manage the early labor (characterized by mild intensity, brief duration, lengthy period, and irregular contractions) and the intermediate labor (characterized by moderate intensity, increasing duration, decreasing period, and progressively more regular contractions) phases so as to minimize their pre-birth presence in a hospital and consequentially maximize their pre-birth experience in a familiar and friendly environment such as their home. It is a concern that they might inadvertently delay going to the hospital for too long and increase the danger of entering active labor (the intense phase when the baby is born) in an uncontrolled situation such as while in transit to the hospital. Further, many mothers prefer to use certain traditional pain management techniques such as visually concentrating on a focus object or physically squeezing an object with their hand to minimize the need for pain reduction medications.

The contraction duration, period, and regularity are important indicators of the immediacy of birth. It is typical that active labor will soon begin when intermediate labor contractions have progressed to where the uterus muscles contract for about a one minute duration, and with a contraction cycle period which is regular and takes about three minute from the start of one contraction until the start of the next contraction. It is typical to seek professional assistance such as going to a hospital prior to the start of active labor. Many doctors and their supportive staff use contraction timing and consistency as an initial measure of a woman's labor progress. Many prefer to not participate until intermediate labor has concluded because they are typically unable to beneficially contribute until active labor begins. It is, consequently, useful for a mother to know the timing characteristics of her contractions to know with some confidence when active labor is nearing and she should seek medical assistance. Due to contraction to contraction variability an average of contraction timing provides a more reliable indicator than any single contraction measurement.

The traditional method of determining the approach of active labor has been with the tabulation of clock or stop watch readings using a pad of paper and pen, and the calculation of contraction duration, period, and the average of both using a calculator. This process presents difficulties because during labor the mother may find these tools and procedures distracting and irritating at a time when she needs to concentrate on contending with pain and is under strong emotional stresses. A support person such as a spouse, if available when the labor process unpredictably begins, needs to pay attention to providing various other kinds of physical and emotional support during intermediate labor, not fumbling with paper, pen, clock, and calculator. The traditional technique is often inaccurate due to errors caused by the commotion that accompanies labor. Reading a clock, tabulating timing data, calculating a contraction duration and period, and analyzing the average contraction timing to discern the variability of the contractions is certainly not what a woman in labor wants to be distracted with, either. Paper, pen, clock, and calculator are often inconvenient due to the context of birth labor such as in bed or a moving vehicle.

An established pain management technique for a woman in labor is to acutely focus her attention on a small distinct object. Common focus objects include a hand held photograph or a feature of the room she is in. Such focus objects are convenient, but they have problems when she changes body orientation or somebody obstructs her view of the object. If she needs to travel, as to the hospital, she may lose access to the focus object. Changing light conditions, such as with darkness or shadows cast in a moving vehicle, may make the focus object indistinct. Lost access to her focus object during contractions introduces a crisis. If the woman prepares for birth with pain management training exercises and she is not able to use the same focus object during birth as she trained with then she will lose those benefits which derive from using the same familiar focus object. A better focus object would be portable, ergonomic, familiar, self illuminating, distinct, under the woman's control, and impart a sense of pro-active participation in managing her pain by responding to actions she take.

Another established technique for a woman to manage labor pain is to squeeze an object. This can be, for instance, a hand of a care giver such as a husband or nurse if they are available. Whatever the woman squeezes must tolerate the considerable pressure of her grip, be portable, be comfortable for the size of her grasp, be accessible as she changes body orientation, be easily sanitized, and be convenient in weight and configuration so that it does not contribute to her discomfort or distraction.

The extreme demands of birth labor make otherwise unremarkable tasks, such as controlling a machine with an inconvenient user interface or the traditional method of measuring and calculating contraction timing, problematic. Traditionally a woman must determine when the active labor phase is nearing and it is time to, for instance, go to the hospital. This normally trivial decision process is made difficult during the stresses of labor because of the intensity of the emotions at play and the debilitating effects of labor pain.

Prior art in birth labor timing apparatus solved some of the problems indicated above, but without an ergonomic interface appropriate to a woman in labor, without the benefit of automated calculations that help measure birth labor progress, without the benefit of an automated alert to the probable start of active labor, and without the benefit of convenient pain management features. The present invention economically, ergonomically, accurately, and conveniently resolves the previously described problems.

### BRIEF SUMMARY OF THE INVENTION

The invention can be embodied as a hand held device to be used during birth labor and during preparatory pain management training exercises. The device can be used for the tracking of birth labor parameters, such as:

1. contraction duration

2. contraction period

3. average contraction duration

4. average contraction period

5. if the average contraction period exceeds a predetermined timing threshold

The invention may provide pain management assistance such as providing a squeezable handle grip and a visual focus object.

Said handle grip may contain an integrated control input means coupled to other circuits. Said control input means may enable the user to synchronize circuit states with said handle grip actuation activity.

Said control input means may be ergonomically configured in a manner sympathetic to the special needs of a person engaged in birth labor. Said control input means may conveniently be flashed, which is to rapidly actuate then de-actuate or de-actuate then actuate the control input means to produce a message symbol known to the algorithmic invention executable firmware codes. Said control input means may be rapidly flashed in a set of multiple flashes to form a message symbol determined by the number of flashes in the set. The meaning of control input means actuation and de-actuation and message symbols may be contextually derived by algorithms performed by the invention executable firmware codes.

Said ergonomic configuration may provide that the handle grip is a size, weight, and external texture which is comfortable and convenient for use by a typical user in a birth labor context. The configuration may further provide that the control input means may be concurrently actuated in a manner which is non-stressful, convenient, and well managed by a typical user during birth labor while the visual outputs of the invention are being conveniently observed. The configuration may further provide that an associated data display means is optimally positioned and oriented relative to a handle grip thus enabling the user to more conveniently view data, semaphore, or focus object display outputs from the invention while holding, squeezing, or controlling the invention by means of the handle grip means and control input means. The configuration may further provide that the handle grip and its attachments be sufficiently robust to well tolerate pain management related squeezing of the handle grip. The configuration may further facilitate actuation of said control input means responsively to user grasping of the handle grip as, for instance, a natural response to birth labor events.

Said visual focus object may be useful is assisting certain pain management procedures. Said usefulness may be increased by synchronizing said visual focus object with the actuation of said control input means.

The invention may provide algorithmic means coupled to a control input means for the user to conveniently select a datum of choice to be displayed on the data and semaphore display means. The datum selected for display may include:

1. a status pattern indicating the device is in Sleep Mode

2. a status pattern indicating the device is in Idle Mode

3. a visible means indicating that the displayed data is of contraction duration type

4. a visible means indicating that the displayed data is of contraction period type

5. the present contraction duration timing data

6. the present contraction period timing data

7. the recent average contraction duration timing data

8. the recent average contraction period timing data

9. a focus object for pain management

10. a status pattern indicating a contraction duration measurement is in progress

11. a status pattern indicating a contraction period measurement is in progress

12. a status semaphore indicating a user Alert (possible start of active labor)

One embodiment of the invention may include algorithmic user control input means for distinguishing between various legitimate and illegitimate user control operations or messages, such as:

1. the legitimate sustained activation of the control input means

2. the legitimate sustained de-activation of the control input means

3. the legitimate flashing of the control input means

4. the legitimate multiple flashing of the control input means

5. all illegitimate manipulations of the control input means

### OBJECTS OF THE INVENTION

The invention provides the integration of numerous functions useful during birth labor, which may include:

1. integration of functions for assisting birth labor into a single device

2. economical configuration of functions for assisting birth labor in a single device

3. economical embodiment of functions for assisting birth labor in a single device

4. ergonomic embodiment of functions for assisting birth labor in a single device

5. improved algorithmic method of a user's control interface

6. improved algorithmic method of control of a device using a single control input

7. improved electrical circuit method for embodying a user's control interface

8. means of integrating a user input means with a device handle means

9. means of integrating a user input means with a pain management means

10. improved method of measuring labor contraction timing data

11. numerical calculation of useful data based on measured labor contraction timing data

12. numerical comparison of labor contraction data and its numerically calculated derivatives to pre-established threshold limits

13. logical comparison of digital data derived from labor contraction

14. automated alerting of a user to a condition determined by processing labor contraction timing data

15. integration of pain management means with a birth labor measurement means

16. synchronization of pain management means with labor measurement means

17. integration of a pain management means with the means for detecting labor contraction events

18. ergonomic configuration of a handheld device concurrently enabling convenient pain management means and convenient data observation means

19. automatic control of a data display to minimize user distractions and accentuate visibility of a data conveying semaphore display

20. improved embodiment of user interface circuits, timing circuits, and data display circuits

21. automatic power management of a labor assistance device so that the user does not need to be concerned about premature battery energy loss prior to the conclusion of a birth event.

### BRIEF DESCRIPTION OF THE SEVERAL VIEWS OF THE DRAWINGS

FIG. 1 is a front and side view of the present invention.

FIG. 2 is a block diagram of a preferred embodiment of the present invention.

FIG. 3 is a schematic diagram of the electrical circuits in a preferred embodiment of the present invention.

FIG. 4 is a block diagram of the variable capacitance actuated control input means in a preferred embodiment of the present invention.

FIG. 5 is a timing diagram of some signals in the variable capacitance actuated control input means in a preferred embodiment of the present invention.

FIG. 6 is a state diagram of the present invention firmware system showing the operational modes in a preferred embodiment of the present invention.

FIG. 7 is a state diagram of the present invention firmware system showing data display operation in a preferred embodiment of the present invention.

### DETAILED DESCRIPTION OF THE INVENTION

While the present invention is described herein for convenience by reference to a particular embodiment other functions and features may also be included, by those skilled in the art which fall within the scope of this invention.

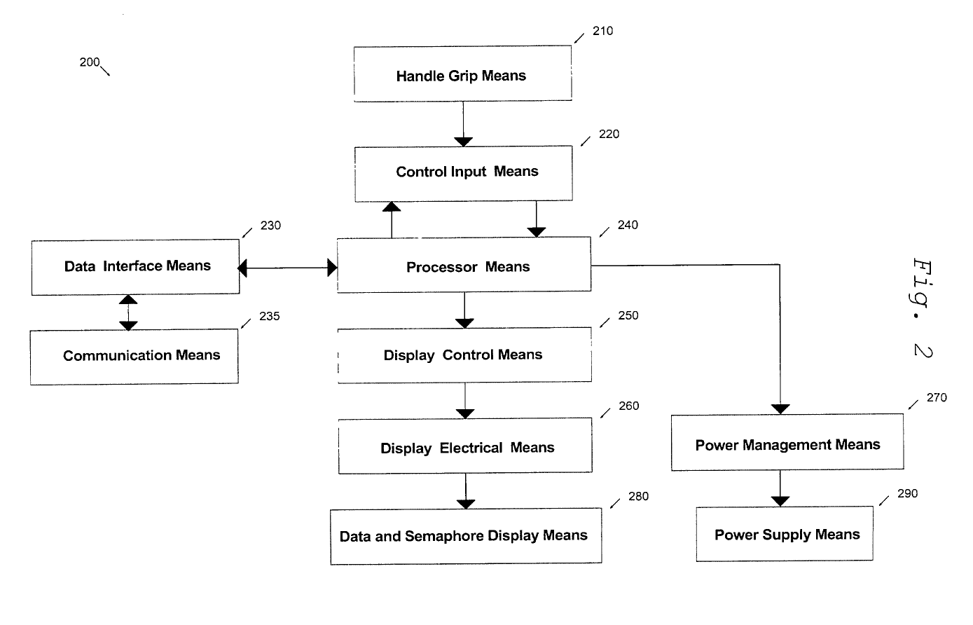

In a preferred embodiment as shown in the system block diagram FIG. 2, the invention may include the novel and advantageous integration 200 of functions described herein for birth labor monitoring and pain management. The invention may include a Handle Grip Means 210 which can contain multiple useful attributes such as physical support, pain management, and an advantageous configuration for the Control Input Means 220. The invention may include a Control Input Means 220 which advantageously communicates user command inputs to the Processor Means with a user actuated control. The invention may include a Data Interface Means 230 allowing the exchange of data between the Processor Means and additional circuits which may be an alternate means of focus object implementation 125 or other data exchanging means Option1. The invention may include a Communication Means 235 enabling the communication of data with external data providing or data receiving entities. The invention includes a Processor Means 240 which may execute the invention firmware or software codes of the invention to sequence device operation responsive to Control Input Means 220, communicate with the Data Interface Means 230, communicate with the Communication Means 235, control the Power Management Means 270, directly or indirectly control the Display Control Means 250, perform event measurements, perform numerical and logical calculations, and control system operation. The invention may include a Display Control Means 250 for controlling a Data and Semaphore Display Means 280 responsively to the Processor Means 240. The invention may include Display Electrical means 260 to electronically actuate the Data and Semaphore Display Means in response to the Display Control Means 250. The invention may include a Power Management Means 270 to conserve energy without disadvantageously requiring a power control switch. The invention may include a Data and Semaphore Display Means 280 for visually outputting information from the device. The invention includes a Power Supply Means 290 which provides operational energy to the device.

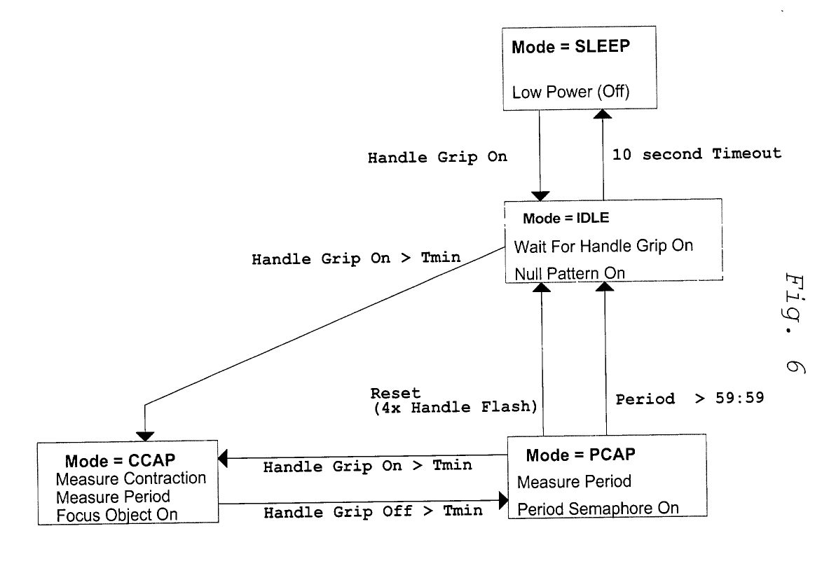

FIG. 6 gives an example of a firmware process in this embodiment showing some possible device modes and conditions for changing modes. Other firmware functions may also be included in the invention as can be programmed for the particular desired functions by one skilled in the art.

In the preferred embodiment if the device is in the Sleep Mode, identifiable by all semaphore lights and the data display OFF, it will enter the Idle Mode when it detects activation of the handle grip. Idle Mode is identifiable by all semaphore lights OFF and the data display shows a distinctive Idle Mode pattern. The device will re-enter Sleep Mode in 10 seconds if there is no further change in handle grip activation state. When in Sleep Mode the device substantially ceases executing invention firmware codes, consequentially reducing battery energy consumption rate to a small fraction of energy consumption in other modes. Sleep Mode approximates the device being OFF without requiring a power switch. While in Sleep Mode the device may periodically and briefly executes invention firmware codes to test for handle grip activation. If the test indicates the handle grip is not actuated the device enters Sleep Mode. The test for handle grip activation is made every approximately 0.017 seconds while in Sleep Mode.

In the preferred embodiment if the handle grip is continuously actuated the device will, after a brief delay to differentiate continuous handle grip activation from noise, errors, or command flashes, begin measuring a new contraction duration and period. Both measurements begin at zero. The device will enter the CCAP Mode and the data display will show the incrementing contraction duration. Similarly, if the handle grip is continuously de-actuated the device will, after a brief delay to differentiate continuous activation from noise, errors, or command flashes, discontinue measuring the present contraction duration and continue measuring the present contraction period. The device will enter the PCAP Mode and the data display will show the present incrementing contraction period. The handle grip may be flashed at any time without changing device mode.

In the preferred embodiment the measurement of a contraction duration and period is evidenced by illumination of the pain management focus object of semaphore light 117 or alternately of semaphore light 125, regardless if the data display is ON or OFF and regardless of what type of data is selected for display. The measurement of a contraction period only is evidenced by illumination of the contraction period semaphore 121, regardless if the data display is ON or OFF and regardless of what type of data is selected for display. When the data display is OFF there is always a semaphore light illuminated to provide visual feedback to the user if the device is measuring a contraction event.

In the preferred embodiment if contraction duration or average contraction duration data is displayed the left most of the four digit data display will be the letter “c”. If contraction period or average contraction period data is displayed the left most of the four digit data display will not be the letter “c”. If any change is made in the handle grip activation the data display will be ON for 10 seconds then turn OFF to conserve battery energy and minimize potential user distraction away from the focus object semaphore light 117 or alternately of semaphore light 125. The data display will blink ON and OFF if the data being displayed is the average contraction duration or average contraction period. The colon semaphore may rapidly blink ON and OFF if the average contraction period is less than the Alert threshold.

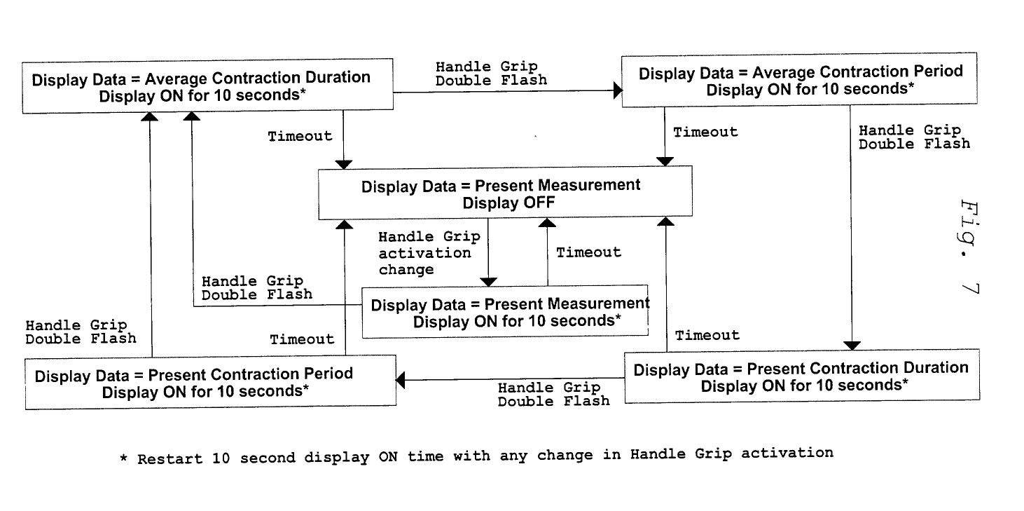

In the preferred embodiment illustrated in FIG. 7 if the handle grip is rapidly flashed twice the data display shows the average contraction duration based on the prior four contraction measurements if they exist. If there have not been four contraction event measurements since the device was reset then an approximation of the average is calculated. If, while the display remains ON, the handle grip is again flashed twice the data display similarly shows the average contraction period or its approximation. If, while the display remains ON, the handle grip is again flashed twice the data display shows the present contraction duration measurement. If, while the display remains ON, the handle grip is again flashed twice the data display shows the present contraction period. If, while the display remains ON, the handle grip is again flashed twice the four state selection method for selecting data to display repeats itself starting with again displaying the average contraction duration. When the data display is allowed to turn OFF due to handle grip inactivity the data selected for display will be the present contraction duration if the device is in CCAP Mode and the present contraction period if the device is in PCAP mode. Contraction event timing is unaffected by handle grip flashing.

In the preferred embodiment the maximum measurable contraction duration is 4:15 minutes and the maximum measurable contraction period is 59:59 minutes. These respective limits exceed normal birth labor event timing. If the device measures a contraction period greater than 59:59 it will assume the device is unused and automatically enter Idle Mode to conserve battery energy.

In the preferred embodiment if the device is in PCAP Mode and the handle grip is rapidly flashed four or more times the device resets itself by clearing data averages and measurements to zero and the device enters Idle Mode.

In the preferred embodiment the colon semaphore in the data display blinks if the average contraction period is less than a preset threshold of three minutes since this suggests active labor may begin soon. This user Alert is intended to draw attention to the need to take certain actions, such as going to a hospital for instance, in preparation for the birth.

In the preferred embodiment simplified operator instructions may be adhesively attached to the rear panel 130 of the device. The instructions may be advantageously available in different languages.

In the preferred embodiment a pain management feature of the device may be a focus object semaphore light which synchronizes with activation of the handle grip. The focus object semaphore light is illuminated when the user actuates the handle grip under the influence of contraction pain, when the focus object is needed, and is un-illuminated when the handle grip is de-actuated between contractions when the focus object is not required.

In the preferred embodiment a pain management feature of the device may be the handle grip which is ergonomically designed to be squeezed during contraction pain. This is advantageously when the handle grip is actuated to measure the contraction duration. The optimized physical configuration allows the handle grip to be comfortably squeezed with great pressure without damaging the device. The optimized physical configuration allows the handle to be squeezed without compromising visual access to the device data display or semaphore lights.

In the preferred embodiment the invention is a hand held appliance 100 of convenient size, weight, configuration, and integrated functions 200 to assist with birth labor.

In the preferred embodiment the Handle Grip Means 210 is a physical structure 140 with an internal cavity 142 for batteries BT1a, BT1b, BT1c, BT1d, an end cap 143 to secure the batteries, a utility strap attachment bracket 145, a finger guide assembly 144 to assist positioning user's finger over conductive electrodes CX1a and CX1b associated with Control Input Means 220. It is robustly re-enforced and attached to top assembly 101.

In the preferred embodiment the Control Input Means 220 is a circuit which allows the MCU (i.e. microcontroller unit) to sense if the user has grasped (actuated) the handle grip. It is explained below in greater detail.

In the preferred embodiment Data Interface Means 230 is a signal port supporting device firmware defined data transfers to an optional circuit or for electrical drive of an electrical load such as an LED which may be alternate focus object element 125.

In the preferred embodiment the Processor Means 240 is U2, a PIC12C509A MCU that includes the clock oscillator, central processing unit, random access data memory, program memory, an instruction program counter with stack, special purpose registers, input/output resources, and special purpose peripheral logic elements for performing event timing, Sleep Mode control, and power on reset.

In the preferred embodiment the Display Control Means 250 provides a multiplicity of logic outputs useful for turning the time multiplexed Data and Semaphore Display Means 280 elements ON and OFF under MCU control. It is comprised of two 74HC595, U1 and U3, serial to parallel shift registers with buffer register outputs. The outputs are sufficiently robust to source or sink current for a seven segment LED display segment or for a semaphore LED. The benefit of using two shift register devices for producing the required logic output signals relative to the obvious technique of utilizing an MCU with a greater number of output pins is economic, since the cost for two shift registers plus a minimal pin count MCU is less than the cost of a higher pin count MCU.

In the preferred embodiment the Display Electrical Means 260 provides current amplification and current limiting functions which allow the data display and semaphore lights to be operated in an economical time multiplexed manner. Current amplification is provided by transistors Q1, Q2, Q3, and Q4. Current limiting is provided by RP1c, RP1d, RP2a, RP2b, RP2c, RP2d, RP3a, RP3b, RP3c, and RP3d.

In the preferred embodiment the Power Management Means 270 is the invention firmware code execution forcing all Data and Semaphore Display Means 280 elements OFF and signal RCL low prior to the device entering Sleep Mode when the device is not in use.

In the preferred embodiment the Data and Semaphore Display Means 280 is multifunction clock seven segment display assembly DSP1. It includes four digits 112, 113, 114, 115 with decimal points 116, 117, 118, 119, a colon 120a and 120b, and an semaphore light 121. The decimal point elements may alternately be used as semaphore elements. It is used in the preferred embodiment because the manufacturer's economy of scale from supplying the clock market makes it very inexpensive relative to competitive display types. An LED display technology is used because the display must be easily visible in low light conditions.

In the preferred embodiment the power supply 290 is four AA cell batteries BT1a, BT1b, BT1c, BT1d providing approximately 6 volts which provide energy to power the system. Typical AA alkaline batteries provide sufficient capacity for at least one complete birth labor procedure.

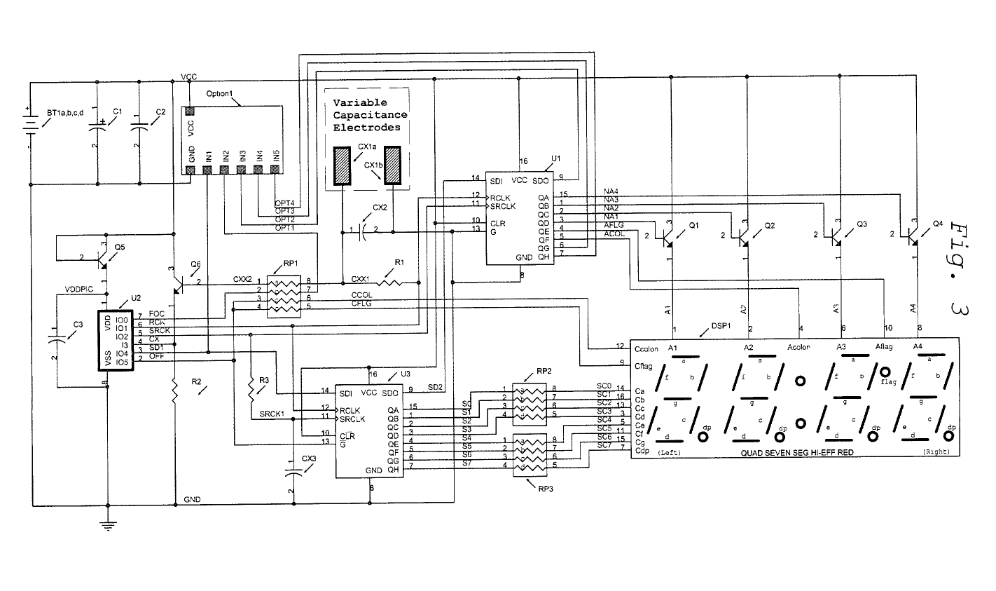

FIG. 3 shows the electrical schematic for a preferred embodiment for the invention.

In the preferred embodiment battery BT1 provides continuous power to the system. Providing continuous power is desirable because when the device is in Sleep Mode the Power Management Means 270 provides power reduction comparable to a switched power system with the power switch turned OFF but with advantageous cost, reliability, and convenience benefits.

In the preferred embodiment capacitors C1 and C2 provide power supply decoupling action which attenuates electrical noise for the benefit of improved MCU U2 and shift register U1 and U3 operation as well as reducing EMI emissions from the circuitry. In the preferred embodiment capacitor C1 is a large capacity electrolytic type which attenuates low frequency noise. Capacitor C2 is a small capacity ceramic type which attenuates high frequency noise. The combined use of the two capacitor types provides superior power supply noise attenuation than either individual type does due to the partial overlap of their respective spectra of highest noise attenuation.

In the preferred embodiment transistor Q5 reduces the power supply voltage VCC to a voltage which is consistent with the MCU U2 manufacturer's specifications. The collector to emitter voltage drop of Q5 may be in the range of 0.5V to 0.7V range for all MCU U2 operational currents so that the nominal 6V power supply voltage will not exceed 5.5V, the manufacturer's specification for maximum operating voltage of MCU U2.

In the preferred embodiment capacitor C3 provides further power supply decoupling for MCU U2. This advantageously enhances the stability and accuracy of the calibrated clock oscillator integrated into MCU U2. This is beneficial because the stability and accuracy of the clock oscillator proportionally affects the stability and accuracy of the system timing functions which are derived from the clock oscillator.

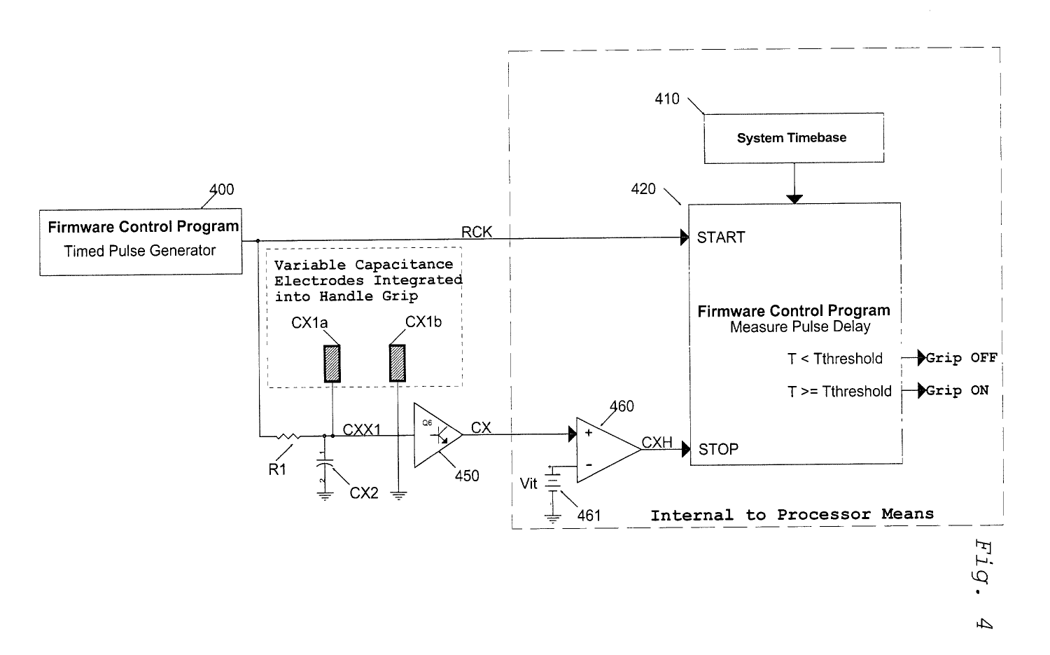

In the preferred embodiment MCU U2 is a self contained microcontroller. It provides such system functions as timebase clock, data calculation, data storage, operational logic, and sequential control signals. It contains read only memory which is programmed with executable invention firmware codes during the manufacturing process. When power is initially applied the MCU U2 resets itself and then executes the invention firmware codes. From time to time the MCU U2 uses the control input circuit (see FIG. 4) to resolve decisions encountered in executing invention firmware codes. From time to time MCU U2 sequentially sends out patterns of control signals and data signals to shift registers U1 and U3 so that data display assembly DSP1 will provide an intended optical display to an observer.

In the preferred embodiment transistor Q6, resistor RP1a, and resistor R2 form a DC level shifting buffer amplifier as part of the Control Input Means 220. A signal CXX1 is essentially replicated as signal CXX2 because there is an insignificant current flowing through RP1a due to the very high input impedance looking into the base of transistor Q6. The purpose of resistor RP1a is to protect transistor Q6 and MCU U2 from possible electrostatic discharge (ESD) damage in the event capacitance sense electrode CX1a receives an ESD event and to prevent Q6 from oscillating. Signal CXX2 is essentially replicated as signal CX by passing from the base of transistor Q6 to the emitter of Q6, except that it is diminished by a silicon junction voltage drop of approximately 0.7 volts when signal CX would be greater than zero volts and otherwise diminishes to approximately zero volts. Transistor Q6 act as a buffer amplifier circuit which usefully presents a very high impedance to signal CXX1 and a low impedance to MCU pin 4, the load of signal CX. The high impedance loading of signal CXX1 prevents problematic signal distortion which might otherwise be caused by leakage currents associated with MCU U2 pin 4 flowing through an advantageously large valued resistor R1. The minimization of MCU U2 leakage current effects on signal CXX1 allows resistor R1 to be of relatively high value to advantageously create a long time constant for practical values of CX1a, CX1b, and CX2. The dynamic operation of the handle grip actuation sense circuit utilizing signals RCK, CXX1, CXX2, and CX is described below.

In the preferred embodiment resistor RP1b is an ESD protection and current limiting resistor for optional data interface Option1, resistor RP1c provides current limiting for the colon semaphore elements 120a and 120b, and resistor RP1d provides current limiting for the flag semaphore element 121.

In the preferred embodiment resistor R3 and CX3, the effective input parasitic capacitance of U3 pin 11, provide a delay of logic clocking signal SRCK produced by MCU U2 pin 5. This delay eliminates a race condition by assuring that shift register U1 will clock data signal SD2 into its first shift register flip flop before output signal SD2 from U3 pin 11 changes. The race condition might otherwise exist if U1 is a slow device, U3 is a fast device, signal SRCK has a slow slew rate, U1 has a high VIH switching threshold, and U3 has a low VIH switching threshold.

In the preferred embodiment shift registers U1 and U3 accept serial data stream SD1 from MCU U2, de-serialize it using synchronous shift register clocks SRCK and SRCK1, synchronously present it to their output ports using buffer register clock RCLK, and drive various data display signals. Data SD1 is shifted from the MCU U2 into U3 on the rising edges of SRCK1. Serial data output from the internal 8 bit shift register in U3 is presented as signal SD2 to the input of the internal shift register in U1. This effectively makes a 16 bit shift register which can be serially filled with data derived by invention firmware code execution in MCU U2. The de-serialized data is transferred from the internal shift registers to the buffer registers and parallel output port pins of U1 and U3 on the rising edge of RCLK. The output pins of U3 become a high impedance, and the display outputs are disabled, if the output enable input on U3 pin 13, signal OFF, goes high. Signals S0 through S7 provide active low segment cathode drive for the LED seven segment displays in DSP1. When any of the signals S0 through S7 are at a logical low state they can sink current from their respective display segments, allowing segments in any DSP1 display digit with its anode pulled high to turn ON. The segment drive current is limited by current limiting resistors RP2a,b,c,d and RP3a,b,c,d to levels appropriate for the specifications of the display. When any of S0 through S7 are at logical high levels their associated segments are turned OFF. Signals ACOL and AFLG enable the colon and flag semaphore LEDs in the display, respectively. When signals NA1 through NA4 are logical high they enable display DSP1 digits 1 through 4 respectively by way of current amplifier transistors Q1 through Q4, respectively. When NA1 through NA4 are high they cause their respective transistor base to go high which pulls the associated transistor emitters to approximately one silicon diode junction drop (0.7V) below VCC. This allows forward biasing of the anodes of the associated DSP1 digit LEDs. The preferred embodiment uses a well known time multiplexed display technique in which only one digit is enabled at a time so that the segment drive signals, S0 through S7, can assume the proper data pattern to produce the desired information display on the enabled digit. A short time later only the next digit anodes will be enabled and the data pattern appropriate to this next digit will be put on S0 through S7 to produce the proper display pattern for that digit. This process continues until all four digits are successively scanned and then the process starts over. If the scanning is sufficiently rapid the observer subjectively sees the display as if all digits are driven continuously. The successive scanning of the DSP1 display digits and the synchronization of the segment drive patterns to the digits is performed under invention firmware code execution in MCU U2.

In the preferred embodiment data display assembly DSP1 contains four seven segment digits with decimal points plus a colon and a semaphore LED. The digits are common anode, meaning that all segments in a given digit have their anodes connected together. The similar segment of all digits have their cathodes connected together.

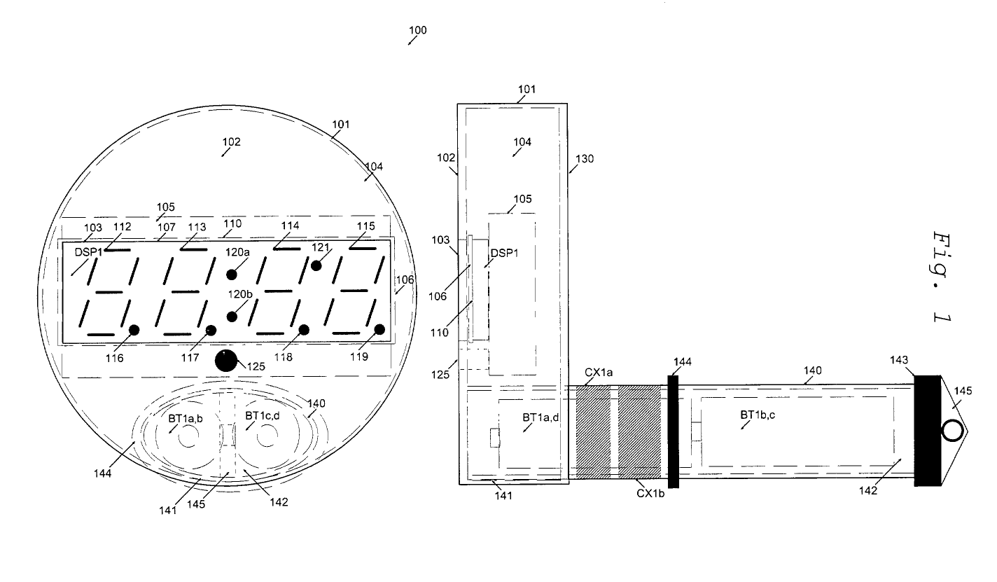

FIG. 1 shows the preferred physical embodiment of the invention. It uses an asymmetrical mushroom shape enclosure comprised of top assembly 101, rear panel 130, and handle grip 140. A cavity 104 internal to the top assembly 101 provides space for electrical components and interconnections 105. The face of the enclosure, 102, is large enough to place display assembly DSP1 inside the enclosure. The display assembly DSP1 is viewable through a rectangular window 103 in front panel 102. A colored semi-transparent optical filter plate 106 is placed over the window to improve subjective data display and semaphore light contrast. The battery BT1 may be placed in the cavity 142 of handle grip 140. Alternatively, the battery BT1 may be placed inside top assembly 101.

In the preferred embodiment the handle grip 140 is attached to the top assembly 102 in a robust and reinforcing manner such as combination butt and lap joint 141 at the inner circumference of top assembly 102. At proximity with the surface of handle grip 140 are contour matching electrode plates CX1a and CX1b which extend around the circumference of handle grip 140 and are ohmically insulated from the user's touch. Electrode plates CX1a and CX1b have a small gap between them so that they are electrically insulated from each other. Electrodes CX1a and CX1b may be of different dimensions, with CX1b being preferentially larger. The dimensions of electrodes CX1a and CX1b are chosen to match the typical dimensions of a human index finger circumferentially curled to overlap them in a natural grasp when the hand palm is placed central to the handle grip 140.

The preferred embodiment advantageously uses a single control input means 220 as shown in FIG. 4. Under invention firmware code execution control the MCU U2 from time to time assures that stimulus signal RCK is driven logically low for much longer than the maximum time constant of sensor resistor R1 and the maximum possible combined capacitance of parasitic capacitance CX2 and sensor electrodes CX1a and CX1b in the actuated condition. Parasitic capacitance CX2 includes the total signal CXX1 node capacitance driven by sensor resistor R1 when there is no activation coupling between electrodes CX1a and CX1b. After signal RCK has been assured low as described above the signal RCK is driven high. With CMOS technology circuits the RCK signal is essentially at ground voltage when driven low and when driven logically high it is essentially at the VCC voltage for the MCU U2. In response to signal RCK transitioning from low to high the signal CXX1 will exhibit a well known inverse exponential voltage rise to asymptotically approach the high RCK voltage. The time that it takes for signal CX to rise to MCU U2 transition threshold voltage Vit on U2 pin 4 depends on the time constant of resistor R1 and the node capacitance of CXX1, which is the parasitic capacitance CX2, and sensor electrode CX1a capacitance. If a user has grasped the handle grip in an actuating manner to couple electrodes CX1a and CX1b by means of her sufficiently conductive finger forming an intermediate capacitive electrode then the capacitance between CX1a and CX1b, and hence the CXX1 node capacitance, will be significantly increased. It is well known that capacitance between two electrodes may be described as C=K*A*(1/d), where C is the capacitance, K is a constant, A is the effective projection area between the two electrodes, and d is the effective distance between the electrodes. Without the user's actuating finger the CX1a capacitance to circuit ground is minimal because of the relatively small projection of its area onto the device's circuit ground area and the relatively large effective separation between them. When a finger overlaps electrodes CX1a and CX1b in an actuating manner it forms a relatively large projection area on both electrodes with a relatively small separation between each electrode and the finger and hence creates a relatively large capacitance between each electrode and the finger. Because the finger is conductive within itself this effectively places in series connection the two capacitances formed between the finger and the two electrodes CX1a and CX1b. Because these two capacitances are relatively large, their series equivalent which is added to CX2 to form the node capacitance of CXX1 is also relatively large. An increased CXX1 node capacitance from an actuated handle grip results in an increased time constant for node CXX1 relative to when the handle grip is not actuated, and hence an increased delay from the rising edge of signal RCK until signal CX exceeds transition threshold Vit. Said delay is readily measured by the executing invention firmware codes and which can thereby determined if the handle grip has been actuated by determining if the delay is greater than or less than a predetermined threshold delay. A delay less than the threshold delay from RCK going high until CX is detected to be high corresponds to the handle grip not actuated, while a delay from RCK going high until CX is detected to be high which is as long or longer than the threshold delay corresponds to the handle grip in an actuated state.

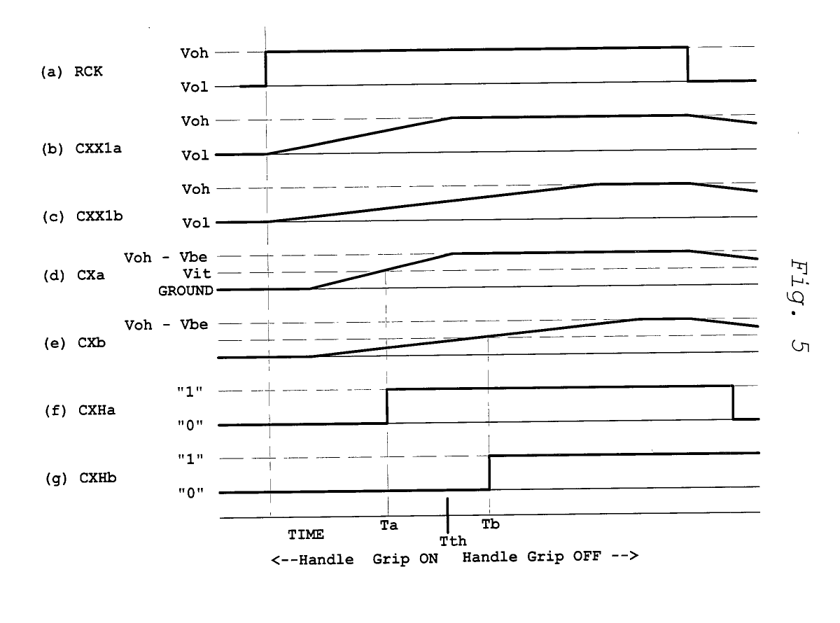

In the preferred embodiment the Control Input Means 220 is shown the schematic of FIG. 3, further in FIG. 4, and is illustrated in the signal timing diagram of FIG. 5. In FIG. 5 signal RCK has been low for a very long time prior to it rising as shown at the left of the diagram. In response to RCK transitioning high the signal CXX1 transitions according to an inverse exponential as shown in CXX1a when the handle grip is unactuated and CXX1b when it is actuated. The slope of CXX1a is initially much faster than for CXX1b. The transistor buffered signals CXa and CXb derived from CXX1a and CXX1b, respectively, show a similar variation in delays between them. The un-actuated handle grip causes CXa to pass through the MCU input transition threshold Vit after time Ta while the actuated handle grip influenced signal CXb requires time Tb to pass through the threshold voltage. The firmware codes use a threshold time Tth to determine if the handle grip is actuated or no, with a shorter RCK high to CXH delay such as Ta determining the handle grip is un-actuated and a longer RCK to CXH delay such as Tb determining the handle grip is actuated.

The preferred embodiment described herein is not the only possible embodiment of the invention. One possible variation is for Data Interface Means 220 or Data Communication Means 235 or circuit Option1 to include a wireless or electrically conductive or fiber optic or telephonic communication means. Said communication means might convey information using a convenient medium, modulation technique, encoding technique, transmission technique, reception technique, demodulation technique, decoding technique, or error correction technique to transmit or receive data over a distance. Said communication means might convey information from a remote data providing means. Said information might describe a physiological change in a person such as temperature or respiration or pulse rate, a body generated voltage or field or sound, a physical location or orientation or movement, a change in body dimension, a change in body size or shape or density or energy conduction, or a change in a sensor reading. Said communication means might convey information to a remote data accepting means such as a computer, a computing device, a visual display, an optical transducer, an acoustic transducer, an electromechanical transducer, a motor, a transducer, a stimulator, an energy radiator, or other device. Said communication means might convey information from a remote data providing means and also to a remote data accepting means. Said communications means might convey messages and control to or from a remotely located person, computer, or machine. Said person may be a medical professional. Said machine may be a robotic entity. Said machine may be a means of transport. Said messages may convey a contextual status, or human executable instructions, or machine executable instructions, or authorizations for actions, or event logging, or status logging, or sensor measurements, or language, or audible signals, or music, or video, or graphic images, or data.

Another alternative embodiment of the invention is to provide a pain or stress management controlling output which is heard, seen, or felt by a person. Said pain or stress management output may be part of an interactive system which is responsive to an input or to temporal events. Said output might include an encoded audio or video or tactile signal which might be heard or seen or felt by a person using a suitable receiver. Said output might be heard or seen or felt by an unaided person. Said output might convey or control sound reproduction equipment. Said output might convey or control a projected or displayed visual pattern using projection and display apparatus. Said output might control an electromechanical, electro-optical, or piezo-electric transducer. Said output might convey a stimulus signal for application to a body to produce a beneficial sensory, physiological, emotional, or psychological reaction. Said output might control a chemical reaction or a modulator of chemical flow.

I claim:

1. a hand held timing device with integrated pain management means comprising:
a handle grip, a control input, a processor, and a display.
